(19) United States
(12) Patent Application Publication
(10) Pub. No.: US 2026/0115247 A1
(43) Pub. Date: Apr. 30, 2026

(54) ETHANOLIC EXTRACT FROM LEAVES OF THE PLANT SALVIA PISIDICA FOR THE TREATMENT OF BORRELIOSIS CAUSED BY BORRELIA SPP

(71) Applicant: PATENTPOOL TARGET GMBH, Muenchen (DE)

(72) Inventors: Renate BURDA, Sauerlach (DE); Jenny MÖRTH-KRETSCHMER, Buchholz in der Nordheide (DE); Elissabeth SAND, Rauenzell/Herrieden (DE); Dirk WEICKMANN, Rauenzell/Herrieden (DE)

(73) Assignee: PATENTPOOL TARGET GMBH, Muenchen (DE)

(21) Appl. No.: 19/142,763

(22) PCT Filed: Oct. 19, 2023
(86) PCT No.: PCT/EP2023/079138

§ 371 Date: Jun. 24, 2025

(30) Foreign Application Priority Data
Dec. 24, 2022 (EP) 22216673.8

(51) Int. Cl. A61K 36/537; A61K 35/644

(52) U.S. Cl. CPC A61K 36/537; A61K 35/644; A61K 2236/333

(57) ABSTRACT

The inventors found in a sage, Salvia pisidica Boiss. & Heldr. substances that can kill Borrelia spp. and Yersinia spp. in vitro and in vivo. Up to now, it has been assumed that a methanol extract or the oil of Silvia pisidica has antimicrobial effects against gram (+) bacteria. Previous experiments confirm this current scientific view.

TBE and FME were described in the 1980s. Tick-borne encephalitis is a disease caused by the TBE virus that occurs with symptoms similar to those of Lyme disease, i.e. fever and partial meningoencephalitis, the inflammation of the brain and meninges.

Lyme disease is caused by bacteria and not by viruses like TBE. The risk of transmission existed “back then” only with every 10,000 or 100,000 tick bites/bites. Nowadays, it can be assumed that Borrelia bacteria are transmitted with every tick bite. The danger of Borrelia and the connection with rheumatic diseases was not recognized for a long time, also because the time between a tick bite and the outbreak of the disease can take up to 40 years. It usually takes months or years after a tick bite before the disease breaks out. Presumably due to the generally weaker immune system of the “modern” population and the general pollution of the entire environment with harmful substances, the outbreak of the disease often occurs much faster today, with more severe courses. We are convinced that most of the rheumatic complaints that occurred in rural populations in the past and still occur today are actually due to Borrelia infections.

Lyme disease initially manifests itself with unspecific general symptoms such as fever, headaches or gastrointestinal complaints. Reddening of the skin (erythema migrans or migratory erythema) is a clear symptom of a Borrelia infection, but does not occur in all Lyme disease infections. The absence of erythema migrans after a tick bite does not automatically mean that no Lyme disease infection has occurred.

Symptoms such as flu, fever and headaches make it difficult to recognize the disease. The organs, joints and muscles as well as the central and peripheral nervous system may be affected. At this stage, the immune system is often no longer able to cope with the infection. Borrelia bacteria only stay in the blood for a short time and settle in the connective tissue and cell walls, they live hidden, so to speak. Here they are difficult to detect and therefore a longer-lasting treatment is necessary.

### State of the Art in Treatment

If the infection is not yet very advanced, it can be treated with antibiotics. The most effective treatment is with pure penicillin (C16H18N2O4S). Whether other antibiotics have a lasting and sustainable effect has not yet been confirmed and is currently still in doubt. However, treatment with penicillin can be helpful if the right time (before the bacteria “hide” in the tissue) is recognized by chance; but recognizing this time is almost impossible. From the time of transmission of the pathogen, a local infection of the skin can occur after a varying incubation period, which becomes visible with a reddening of the skin at the site of the bite. In some cases, the incubation period can last several years, which is why the associated joint pain has long been mistaken for rheumatic complaints and treated unsuccessfully with cortisone ointment. We are convinced that most of the rheumatic complaints that occurred in the past and still occur today, especially in the rural population, are due to Borrelia.

### Problems with Diagnosis and Therapy

Contrary to conventional wisdom, borreliosis can spread very quickly. There is no absolutely predictable course for Lyme disease, as the classic classification into three stages suggests, and the course of the disease does not develop according to law. If the symptoms of the affected person do not fit into the conventional medical classification of Lyme disease, Lyme disease is often ruled out and the symptoms are treated incorrectly. A Lyme disease infection does not follow any pattern! It is not possible to predict how Lyme disease will develop. The symptoms can develop quickly and severely or they can manifest themselves gradually and slowly.

If Lyme disease is not treated in time, it can become a chronic infection. This means that the disease recurs or gets progressively worse. Even years of symptom-free latency periods with subsequent reactivation are possible. The pathogens trigger various clinical pictures. The joints are usually affected, but it can also lead to heart problems and vascular inflammation. Mixed forms of the diseases are also possible Many Lyme disease patients complain of exhaustion and chronic fatigue, which cannot be compensated for by sufficient sleep.

A Lyme disease vaccine suitable for humans is not expected in the near future. In the USA, a corresponding vaccine was withdrawn from the market in 2002 due to alleged vaccination complications. However, this vaccine was only suitable for the American strain of the pathogen and therefore not applicable in Europe.

The only truly effective antibiotic is purely biological penicillin. In our experience, this must be administered no later than six weeks after the infection. This only happens in very few cases.

### Lyme Disease Topical:

The latest studies show that around one in seven people worldwide has already been infected with Lyme disease at least once. According to a study published in BMJ Global Health, Central Europe has the highest infection rate at 20%. It is therefore the object of this invention to develop an effective therapy against Lyme disease. With patent specification WO2007060252A3, we have already had a therapeutic agent against Lyme disease since 2006 which has also been positively evaluated in the context of permissible medical treatment trials. Here we found and developed active substances from total venom cocktails of various myriapods. The disadvantage of this treatment option is the relatively poor availability of the active ingredients. In the present invention, we describe an effective treatment option for Lyme disease with the best availability of the active ingredients.

It is thus a task of the present invention to propose a method for the preparation of a therapeutic composition as well as the composition and an apparatus for its preparation.

The task is solved by the independent claims. Further advantageous embodiments are given in the subclaims.

### Composition and Preparation According to the Invention

The inventors now found in a sage, the Salvia pisidica Boiss. & Heldr. substances that can kill Borrelia spp. and Yersinia spp. in vitro and in vivo. So far it has been assumed that a methanol extract or the oil of Silvia pisidica has antimicrobial effects against gram (+) bacteria. There is hardly any evidence of an effect against gram (−) bacteria. Our tests to date confirm this current scientific view.

The inventors have now discovered that an ethanol extract of the fresh leaves of Salvia pisidica incorporated into honey can kill gram (−) bacteria, especially Borrelia spp. and/or Yersinia spp.

Thus, an ethanolic extract of Salvia pisidica incorporated into honey is suitable for the treatment of Lyme disease.

Around 900 species of sage are known worldwide. Many have pharmaceutically and medically interesting substances. Only a few species have been studied so far. We examined extracts of the following sage species with regard to their Borrelia-killing effect:
Salvia mellifera
Salvia nevadensis
Salvia fruticosa
Salvia cinerea
Salvia pisidica
Salvia lavandulifolia

| | Borrelia burgdorferi | Borrelia afzelli |
|---|---|---|
| Salvia mellifera | − | − |
| Salivia nevadensis | + | − |
| Salvia fruticosa | − | − |
| Salvia cinerea | − | + |
| Salvia pisidica | + | + |
| Salvia lavandulifolia | − | − |

Only extracts of Salvia pisidica showed a demonstrable effect against Borrelia according to the invention:

According to one aspect of the present invention, the composition according to the invention is prepared in the following manner:
a) A certain quantity of leaves, preferably fresh leaves of Salvia pisidica, is placed in a certain quantity of 96% ethanol, or preferably 100% ethanol.
b) The extract from a) is left to stand for at least 24 hours at temperatures of preferably 25 degrees Celsius. The leaves are then filtered off.
c) This active extract from b) is incorporated into 150 ml of liquefied organic blossom honey with constant stirring.
   Cultivation of Salvia pisidica for Optimal Synthesis of Active Ingredients:

In order for plants, especially the species Salvia pisidica required for the use according to the invention, to produce relevant amounts of their active ingredients, it is important to have the right growing media/soil for the plants.

The inventors discovered that Salvia pisidica not only requires a well-drained soil, but that the species also needs a calcareous soil to synthesize its active ingredients. We prefer to use lime soil from flora montana with approx. 20% weathered limestone.

More than 35 different components can be detected in the ethanol extract of Salvia pisidica. Bile acid equivalents, rutin equivalents and catechin equivalents can be increased by using the right substrate. Cultivation in plant containers is more efficient than cultivation directly in the soil. For the application according to the invention, it is important to use the fresh leaves of the plants without stems. We have observed effects at the following concentrations:

| | Ethanol 96% | Ethanol 100% |
|---|---|---|
| 2.5 g/100 ml | Borrelia 20% | Borrelia 20% |
| 5.0 g/100 ml | Borrelia 70% | Borrelia 80% |
| 7.5 g/100 ml | Borrelia 90% | Borrelia 100% |
| 10.0 g/100 ml | Borreliia 100% | Borrelia 100% |

Therefore, the higher concentrations and the extracts in 100% ethanol are the most effective.

### Dosage Forms:

According to the invention, it is shown that the extracts can be taken orally. The active ingredient is absorbed via the digestive tract. The ethanolic extract is particularly easily soluble in honey, so in addition to the direct, pure ingestion of the ethanolic extract, it also makes sense to dissolve it in honey.

Aspects of the present invention:
Ethanolic extract of the leaves of Salvia pisidica to combat Lyme disease:
   At least leaves of Salvia pisidica;
   At least 96% ethanol; and
   At least bee honey.
Ethanolic extract of the leaves of Salvia pisidica to combat Lyme disease with the following ingredients:
a) At least young leaves of Salvia
b) At least 96% ethanol
c) At least bee honey
Ethanolic extract of the leaves of Salvia pisidica to combat Lyme disease with the following ingredients:
a) At least leaves of Salvia pisidica
b) At least 100% ethanol
c) At least bee honey
An ethanolic extract of the leaves of Salvia pisidica for combating Lyme disease according to any one of claims 1 to 4, characterized in that the bee honey has the quality of organic blossom honey.
Process for the preparation of a therapeutic composition according to any one of claims 1 to 5, characterized in that the following measures are taken.
   A certain amount of leaves, preferably fresh leaves of Salvia pisidica, is introduced into a certain amount of 96% ethanol, or preferably 100% ethanol.
   The extract from a) is left to stand for at least 24 hours at temperatures of preferably 25 degrees Celsius. The leaves are then filtered off
   This active extract from b) is incorporated into 150 ml of liquefied organic blossom honey with constant stirring.

The leaves of Salvia pisidica, for use according to the invention, are the most substantial with a length of about 1.7 to 2.1 cm and a width of 0.9 to 1.3 cm (individual leaflets 0.3 to 0.5 cm). Fresh leaves of Salvia pisidica are used, not necessarily young leaves. This means that the leaves are taken directly from the plant and should be processed immediately after harvesting, or after 50 hours at the latest. Directly means without further processing. If they are not processed directly, they should be stored in the dark and at temperatures of 15 to 22 degrees Celsius. Fresh, young, light green leaves, for use according to the invention, have even more active ingredient. These leaves are up to 30 days old-depending on the location of the respective plant.

According to the invention, a kit of parts can be present as a kit, a device and/or a system arrangement.

According to the invention, it is particularly advantageous that the method can be used to operate the proposed devices and units. Furthermore, the proposed devices and units are suitable for carrying out the method according to the invention. Thus, in each case the device implements structural features which are suitable for carrying out the corresponding method. However, the structural features can also be designed as method steps. The proposed method also provides steps for implementing the function of the structural features.

1. A composition:
an extract made with at least 96% ethanol from the leaves of Salvia pisidica grown on permeable calcareous soil; and bee honey.

2. The composition according to claim 1, wherein the leaves have a length of 1.7 to 2.1 cm and a width of 0.9 to 1.3 cm.

3. The composition according to claim 1, wherein the ethanol is 100% ethanol.

4. The composition according to claim 1, characterized in that the bee honey has the quality of organic blossom honey.

5. A process for the preparation of a therapeutic composition according to claim 1, comprising the steps of:
Incorporating a predetermined amount of leaves of the plant Salvia pisidica grown on permeable calcareous soil into a predetermined amount of at least 96% ethanol to produce a decoction;
Leave the brew to stand for at least 24 hours
Filtering the leaves from the brew; and
Stir the decoction into liquefied blossom honey.

6. The process according to claim 5, characterized in that the ethanol is 100% ethanol.

7. A method according to claim 5, characterized in that fresh leaves are used.

8. The process according to claim 5, characterized in that the brew is left to stand at at least 25 degrees Celsius.

9. (canceled)

10. The process according to claim 5, characterized in that the blossom honey is of organic quality.

11-13. (canceled)
